(19) United States
(12) Patent Application Publication
(10) Pub. No.: US 2026/0061010 A1
(43) Pub. Date: Mar. 5, 2026

(54) USE OF BACTERIAL STRAIN FOR ENHANCING 5-HYDROXYTRYPTOPHAN SECRETION BY ENTEROCHROMAFFIN CELL

(71) Applicant: GLAC BIOTECH CO, LTD., Tainan (TW)

(72) Inventors: HSIEH-HSUN HO, Tainan (TW); YI-WEI KUO, Tainan (TW); JIA-HUNG LIN, Tainan (TW); SHIN-YU TSAI, Tainan (TW); CHING-MIN LI, Tainan (TW); YU-CHIEH HSU, Tainan (TW); KO-CHIANG HSIA, Tainan (TW); JUI-FEN CHEN, Tainan (TW); YEN-YU HUANG, Tainan (TW)

(21) Appl. No.: 18/818,794

(22) Filed: Aug. 29, 2024

(30) Foreign Application Priority Data
Nov. 28, 2023 (TW) 112146140

(51) Int. Cl. A61K 35/745; A61K 35/747

(52) U.S. Cl. CPC A61K 35/745; A61K 35/747

(57) ABSTRACT

A method for enhancing 5-hydroxytryptophan secretion by enterochromaffin cells is disclosed, and the method comprises: administering a composition to a subject in need thereof, the composition comprising: a Bifidobacterium animalis subsp. lactis BB-115 strain. Herein, another method for enhancing 5-hydroxytryptophan secretion by enterochromaffin cells through administering a composition to a subject in need thereof, the composition comprising: a Bifidobacterium animalis subsp. lactis BB-115 strain, a Lactobacillus rhamnosus MP108 strain, and a Bifidobacterium longum subsp. infantis BLI-02 strain.

### CROSS REFERENCE

This non-provisional application claims priority of Taiwan Invention patent application No. 112146140, filed on Nov. 28, 2023, the contents thereof are incorporated by reference herein.

### FIELD OF THE INVENTION

The present invention is related to the use of a bacterial strain, and more particularly to the use of a bacterial strain for enhancing 5-hydroxytryptophan secretion by enterochromaffin cells.

### BACKGROUND OF THE INVENTION

Serotonin (5-hydroxytryptamine) can affect humans' emotional stability, happiness, concentration, and sleep quality; therefore, maintaining the serotonin concentration in the body is important for physical and mental health.

Enterochromaffin cells are a subtype of enteroendocrine cells, constituting less than 1% of the total intestinal epithelium cells but producing more than 90% of the body's serotonin. Enterochromaffin cells can directly contact gut microbiota and its metabolites located at a side of the intestinal lumen, and interact with afferent nerve endings and efferent nerve endings located in the lamina propria via synapse connection. Enterochromaffin cells are electro-excitatory and can express functional voltage-gated Na⁺ and Ca²⁺ channels. Accordingly, enterochromaffin cells are similar to primary sensory cells. The aforementioned characteristics enable enterochromaffin cells to serve as bidirectional communication mediators between the intestinal lumen, the intestinal epithelial cells, and the specific primary afferent nerve fibers. It is also believed that enterochromaffin cells are involved in the interactions of the microbiome-gut-brain axis in human cognition, depression, and chronic pain.

Enterochromaffin cells have expression of tryptophan hydroxylase 1, which can convert L-tryptophan into 5-hydrotryptophan. 5-hydrotryptophan can pass through the blood-brain barrier via the systemic circulatory system, and be converted into the serotonin precursor in the brain. Tryptophan is an essential amino acid that the human body cannot synthesize on its own and must be acquired through the consumption of foods rich in tryptophan. Insufficient intake of tryptophan-containing foods or decreased serotonin secretion in the body can lead to various physical and mental disorders, such as anxiety, depression, emotional instability, lack of concentration, and sleep disturbances.

Currently, it is recommended to consume foods rich in tryptophan or take supplements containing 5-hydroxytryptophan to increase serotonin levels in the body. However, excessive supplementation of 5-hydroxytryptophan can cause side effects, such as nausea, diarrhea, and serotonin syndrome. Therefore, there is a need in this field to developing methods for effectively enhancing the secretion of 5-hydroxytryptophan by enterochromaffin cells without causing side effects.

### SUMMARY OF THE INVENTION

In this context, experimental results show that a Bifidobacterium animalis subsp. lactis BB-115 strain exhibits superior activity in enhancing the secretion of 5-hydroxytryptophan by enterochromaffin cells compared to other strains, thereby increasing the precursor substance of serotonin in the brain. As such, it is expected that a Bifidobacterium animalis subsp. lactis BB-115 strain can be used in the application for enhancing the secretion of 5-hydroxytryptophan by the body.

An objective of the present invention is to provide a method for enhancing 5-hydroxytryptophan secretion by enterochromaffin cells, the method comprising: administering a composition to a subject in need thereof, the composition comprising: a Bifidobacterium animalis subsp. lactis BB-115 strain deposited at the China General Microbiological Culture Collection Center with a deposition number of CGMCC 21840.

Preferably, the Bifidobacterium animalis subsp. lactis BB-115 strain is an active strain or an inactivated strain.

Preferably, the method is further for enhancing immunity, treating insomnia, treating depression, treating hypertension, improving somnipathy, improving anxiety, improving inattention, treating menopausal syndrome, or treating gastrointestinal disease.

Preferably, the composition is a medicine, a food, or a health supplement.

Another objective of the present invention is to provide a method for enhancing 5-hydroxytryptophan secretion by enterochromaffin cells, the method comprising: administering a composition to a subject in need thereof, the composition comprising: a Bifidobacterium animalis subsp. lactis BB-115 strain deposited at the China General Microbiological Culture Collection Center with a deposition number of CGMCC 21840, a Lactobacillus rhamnosus MP108 strain deposited at the China General Microbiological Culture Collection Center with a deposition number of CGMCC 21225, and a Bifidobacterium longum subsp. infantis BLI-02 strain deposited at the China General Microbiological Culture Collection Center with a deposition number of CGMCC 15212.

Preferably, the Bifidobacterium animalis subsp. lactis BB-115 strain, the Lactobacillus rhamnosus MP108 strain, and the Bifidobacterium longum subsp. infantis BLI-02 strain are individually an active strain or an inactivated strain.

Preferably, a CFU (colony forming unit) ratio of the Bifidobacterium animalis subsp. lactis BB-115 strain to the Lactobacillus rhamnosus MP108 strain to the Bifidobacterium longum subsp. infantis BLI-02 strain is (1 to 2): 1:1.

Preferably, the CFU (colony forming unit) ratio of the Bifidobacterium animalis subsp. lactis BB-115 strain to the Lactobacillus rhamnosus MP108 strain to the Bifidobacterium longum subsp. infantis BLI-02 strain is 2:1:1.

Preferably, the method is further for enhancing immunity, treating insomnia, treating depression, treating hypertension, improving somnipathy, improving anxiety, improving inattention, treating menopausal syndrome, or treating gastrointestinal disease.

Preferably, the composition is a medicine, a food, or a health supplement.

### BRIEF DESCRIPTION OF THE DRAWINGS

FIG. 1 shows the 5-hydroxytryptophan expression levels of rat pancreatic islet tumor RIN-14B cells with different treatments; wherein * indicates P<0.05, ** indicates P<0.01, *** indicates P<0.001, and **** indicates P<0.0001.

### DETAILED DESCRIPTION OF THE INVENTION

The detailed description and preferred embodiments of the invention will be set forth in the following content, and provided for people skilled in the art to understand the characteristics of the invention.

A vast and diverse population of microorganisms, which are closely linked to health and disease, inhabit mammalian intestines. Microbiome imbalance is associated with the onset of various diseases. For example, autism, neurodegenerative diseases, and schizophrenia are found to be accompanied by gastrointestinal pathology and dysbiosis. Studies show that the composition of microbiota can regulate serotonin levels in the blood. Generally, mice with normal gut microbiota have higher blood serotonin levels compared to germ-free mice. Compared to rats with normal gut microbiota, germ-free rats have larger enterochromaffin cells in their intestines, indicating that the composition of gut microbiota may influence the development or function of these cells.

It is well known that lactic acid bacteria are safe and widely-used probiotics. Common lactic acid bacteria belong to Lactobacillus, Lactococcus, Pediococcus, Enterococcus, Streptococcus, Bifidobacterium, Bacillus, or Leuconostoc.

Studies investigate the application of lactic acid bacterial strains in enhancing the secretion of 5-hydroxytryptophan by enterochromaffin cells. In Food Funct. 2019 Nov. 1; 10(11):7588-7598, the effects of various bacterial strains on the secretion of 5-hydroxytryptophan by rat pancreatic islet tumor RIN-14B cells are tested. It is found that a Bifidobacterium longum subsp. infantis CCFM687 strain can enhance the 5-hydroxytryptophan secretion and reduce depressive behavior in mice; however, Bifidobacterium longum HAN4-25, HUB2-25, GX17-A9, FSHHK25M1, and FSHHK27M1 strains don't show the activity in promoting 5-hydroxytryptophan secretion. Front Microbiol. 2023 May 10:14:1174800 proposes probiotics related to the prevention and treatment of depression. Specifically, a Bifidobacterium longum R0175 strain, a Bifidobacterium infantis 35624 strain, a Lactobacillus helveticus R0052 strain, a Bifidobacterium adolescentis NK98 strain, and Bifidobacterium breve M-16V and M2CF22M7 strains have a positive impact on the treatment of depression. The antidepressant mechanism of bacterial strains may include the regulation of inflammatory responses, tryptophan metabolism, serotonin synthesis, and the hypothalamus-pituitary-adrenal axis through the gut-brain axis.

The foregoing explanation indicates that it is not reasonable to expect that all strains belonging to the same species have the ability to increase 5-hydroxytryptophan secretion. That is, the ability to increase 5-hydroxytryptophan secretion is strain-specific. Lactic acid bacteria are generally recognized as safe strains, and psychological disorders are considered modern diseases. Therefore, achieving applications of lactic acid bacteria for psychological disorders is one of actively pursued objectives.

In a first embodiment of the present invention, a method for enhancing 5-hydroxytryptophan secretion by enterochromaffin cells is provided, and the method comprises: administering a composition to a subject in need thereof, the composition comprising: a Bifidobacterium animalis subsp. lactis BB-115 strain deposited at the China General Microbiological Culture Collection Center with a deposition number of CGMCC 21840.

The BB-115 strain may be an active strain or an inactivated strain, and can be obtained by incubation in medium suitable for growth. The medium suitable for growth is well known to those skilled in the art, and may be, for example, commercially available or self-prepared. The medium suitable for growth may be MRS broth medium, MRS broth medium supplemented with cysteine, and medium supplemented with glucose, yeast extract, and soy peptone. The incubation is performed following procedures well known to those skilled in the art, e.g., New Microbiol. 2013 April; 36 (2): 167-79. Before incubation, the BB-115 strain may be seeded into medium at a bacterial count concentration of 10⁶ to 10¹² CFU/mL, preferably at a bacterial count concentration of 10⁶ to 10¹⁰ CFU/mL, more preferably at a bacterial count concentration of 2×10⁷ to 10⁸ CFU/mL. During incubation, the BB-115 strain is placed at a temperature of 25 to 40° C. for 24 to 48 hours, preferably at a temperature of 37° C. for 24 hours.

In the method, the composition is administrated to the subject in need of enhancing 5-hydroxytryptophan secretion by enterochromaffin cells to increase the 5-hydroxytryptophan level in the body. The subject may be a mammal; the mammal may be a primate, a cat, a dog, a mouse, a rat, a rabbit, a cow, a horse, a goat, a sheep, or a pig; the primate may be a chimpanzee, a human, a gorilla, a bonobo, an orangutan, or a monkey. Further, the composition may be administered to the subject at a total bacterial count of the BB-115 strain of 10⁶ to 10¹⁰ CFU of body weight of the subject per day, preferably at a total bacterial count of the BB-115 strain of 10⁷ to 10⁹ CFU of body weight of the subject per day, and more preferably at a total bacterial count of the BB-115 strain of 10⁸ CFU of body weight of the subject per day. Based on the characteristic of the BB-115 strain to enhance the secretion of 5-hydroxytryptophan by enterochromaffin cells, the method may be provided further for enhancing immunity, treating insomnia, treating depression, treating hypertension, improving somnipathy, improving anxiety, improving inattention, treating menopausal syndrome, or treating gastrointestinal disease.

The composition may further comprise a physiologically acceptable excipient, diluent, or carrier, and the composition may be a medicine, a food, or a health supplement.

On the condition that the composition is a medicine or a health supplement, the physiologically acceptable excipient, diluent, or carrier may be a pharmaceutically acceptable excipient, diluent, or carrier, and may be a solvent, a buffer agent, an emulsifying agent, a suspending agent, a decomposing agent, a disintegrant, a dispersant, a binder, a stabilizing agent, a chelating agent, a gelling agent, a humectant, a lubricant, an absorption delaying agent, or a liposome. Based on this, the medicine or the health supplement may be in an appropriate dosage form, e.g. powder, tablet, caplet, lozenge, pill, capsule, solution, suspension, emulsion, syrup, elixir, concentrate, drop, gel, ointment, spray, or cream. Based on this, the medicine or the health supplement may be administered to the subject via an appropriate route, e.g., oral administration, sublingual administration, intrarectal administration, intranasal administration, intravaginal administration, intraperitoneal administration, percutaneous administration, epidermal administration, intraarticular administration, intraocular administration, or topical administration.

On the condition that the composition is a food or a health supplement, the physiologically acceptable excipient, diluent, or carrier may be a food-acceptable excipient, diluent, or carrier, and may be a fluid milk (e.g., a milk or a condensed milk), a fermented milk (e.g., a soured milk), a milk powder, an ice cream, a cheese, a cottage cheese, a soy milk, a fermented soy milk, a vegetable juice, a fruit juice, a sports drink, a jelly, a cookie, an energy bar, a health food, an animal feed, or a dietary supplement.

In a second embodiment of the present invention, a method for enhancing 5-hydroxytryptophan secretion by enterochromaffin cells is provided, and the method comprises: administering a composition to a subject in need thereof, the composition comprising: a Bifidobacterium animalis subsp. lactis BB-115 strain deposited at the China General Microbiological Culture Collection Center with a deposition number of CGMCC 21840, a Lactobacillus rhamnosus MP108 strain deposited at the China General Microbiological Culture Collection Center with a deposition number of CGMCC 21225, and a Bifidobacterium longum subsp. infantis BLI-02 strain deposited at the China General Microbiological Culture Collection Center with a deposition number of CGMCC 15212. A CFU ratio of the BB-115 strain to the MP108 strain to the BLI-02 strain may be (1 to 2): 1:1, preferably 2:1:1.

The BB-115 strain may be an active strain or an inactivated strain, the MP108 strain may be an active strain or an inactivated strain, and the BLI-02 strain may be an active strain or an inactivated strain. Each bacterial strain can be obtained by incubation in medium suitable for growth. The medium suitable for growth is well known to those skilled in the art, and may be, for example, commercially available or self-prepared. The medium suitable for growth may be MRS broth medium, MRS broth medium supplemented with cysteine, and medium supplemented with glucose, yeast extract, and soy peptone. The incubation is performed following procedures well known to those skilled in the art, e.g., New Microbiol. 2013 April; 36 (2): 167-79. Before incubation, each bacterial strain may be seeded into medium at a bacterial count concentration of 10⁶ to 10¹² CFU/mL, preferably at a bacterial count concentration of 10⁶ to 10¹⁰ CFU/mL, more preferably at a bacterial count concentration of 2×10⁷ to 10⁸ CFU/mL. During incubation, each bacterial strain is placed at a temperature of 25 to 40° C. for 24 to 48 hours, preferably at a temperature of 37° C. for 24 hours.

In the method, the composition is administrated to the subject in need of enhancing 5-hydroxytryptophan secretion by enterochromaffin cells to increase the 5-hydroxytryptophan level in the body. The subject may be a mammal; the mammal may be a primate, a cat, a dog, a mouse, a rat, a rabbit, a cow, a horse, a goat, a sheep, or a pig; the primate may be a chimpanzee, a human, a gorilla, a bonobo, an orangutan, or a monkey. Further, the composition may be administered to the subject at a total bacterial count of the BB-115 strain, the MP108 strain, and the BLI-02 strain of 10⁶ to 10¹⁰ CFU of body weight of the subject per day, preferably at a total bacterial count of the BB-115 strain, the MP108 strain, and the BLI-02 strain of 10⁷ to 10⁹ CFU of body weight of the subject per day, and more preferably at a total bacterial count of the BB-115 strain, the MP108 strain, and the BLI-02 strain of 10⁸ CFU of body weight of the subject per day. Based on the characteristic of the totality of the BB-115 strain, the MP108 strain, and the BLI-02 strain to enhance the secretion of 5-hydroxytryptophan by enterochromaffin cells, the method may be provided further for enhancing immunity, treating insomnia, treating depression, treating hypertension, improving somnipathy, improving anxiety, improving inattention, treating menopausal syndrome, or treating gastrointestinal disease.

The composition may further comprise a physiologically acceptable excipient, diluent, or carrier, and the composition may be a medicine, a food, or a health supplement.

On the condition that the composition is a medicine or a health supplement, the physiologically acceptable excipient, diluent, or carrier may be a pharmaceutically acceptable excipient, diluent, or carrier, and may be a solvent, a buffer agent, an emulsifying agent, a suspending agent, a decomposing agent, a disintegrant, a dispersant, a binder, a stabilizing agent, a chelating agent, a gelling agent, a humectant, a lubricant, an absorption delaying agent, or a liposome. Based on this, the medicine or the health supplement may be in an appropriate dosage form, e.g. powder, tablet, caplet, lozenge, pill, capsule, solution, suspension, emulsion, syrup, elixir, concentrate, drop, gel, ointment, spray, or cream. Based on this, the medicine or the health supplement may be administered to the subject via an appropriate route, e.g., oral administration, sublingual administration, intrarectal administration, intranasal administration, intravaginal administration, intraperitoneal administration, percutaneous administration, epidermal administration, intraarticular administration, intraocular administration, or topical administration.

On the condition that the composition is a food or a health supplement, the physiologically acceptable excipient, diluent, or carrier may be a food-acceptable excipient, diluent, or carrier, and may be a fluid milk (e.g., a milk or a condensed milk), a fermented milk (e.g., a soured milk), a milk powder, an ice cream, a cheese, a cottage cheese, a soy milk, a fermented soy milk, a vegetable juice, a fruit juice, a sports drink, a jelly, a cookie, an energy bar, a health food, an animal feed, or a dietary supplement.

The following examples are offered for further illustrating the invention.

### Example 1: Cell Collection and Incubation

Proc Natl Acad Sci USA. 2009 Mar. 3; 106(9):3408-13 suggests that rat pancreatic islet tumor RIN-14B cells have similar functions to enterochromaffin cells, and thus rat pancreatic islet tumor RIN-14B cells can be used as a model for studying the functions of enterochromaffin cells.

Rat pancreatic islet tumor RIN-14B cells were purchased from the American Type Culture Collection (ATCC). The rat pancreatic islet tumor RIN-14B cells were seeded into RPMI-1640 medium supplemented with 10% fetal bovine serum (FBS) and 1% penicillin-streptomycin, and placed in an incubator with 5% CO₂ at 37° C. for incubation. Afterwards, the culture medium was changed every 2 to 3 days, and the cell were subcultured when they reached 80% to 90% confluence.

### Example 2: Bacterial Strain Collection

The bacterial strains used for function assays are all deposited at the China General Microbiological Culture Collection Center in No. 3, No. 1 Courtyard, Beichen West Road, Chaoyang District, Beijing City, China according to the Budapest treaty. The deposition information is listed in Table 1 below.

TABLE 1
deposition information

| | Deposition | Deposition |
|---|---|---|
| Strain | number | date |
| Bifidobacterium animalis subsp. lactis | CGMCC 21840 | Feb. 26, 2021 |
| BB-115 | | |
| Lactobacillus rhamnosus MP108 | CGMCC 21225 | Nov. 23, 2020 |
| Bifidobacterium longum subsp. infantis | CGMCC 15212 | Jan. 15, 2018 |
| BLI-02 | | |

The following bacterial strains are used as control strains: a Bifidobacterium animalis subsp. lactis HN019 strain (available from Fonterra Co-operative Group Limited), a Lactobacillus rhamnosus LGG strain (available from Chr. Hansen A/S), and a Bifidobacterium longum subsp. infantis M-63 strain (available from Morinaga Milk Industry Co., Ltd.).

### Example 3: Bacterial Strain Incubation

Bacterial strains were seeded into MRS broth medium, and then placed in an incubator with 5% CO₂ at 37° C. for 24 hours to activate strains. Afterwards, the activated strains were seeded into MRS broth medium at a concentration of 2 v/v %, and incubated in an incubator with 5% CO₂ at 37° C. for 24 hours. Then, the resulting culture was centrifuged at 3,000 rpm for 10 minutes at 4° C. to precipitate bacterial bodies. After removing the supernatant, the precipitate was suspended with RPMI medium supplemented with 10% fetal bovine serum, 2 μM fluoxetine, and 1% penicillin-streptomycin to obtain a bacteria solution with an approximate concentration.

### Example 4: Statistical Analysis

All experimental data were expressed as “mean±standard deviation”. Differences between experimental data were assessed using the Student's t-test.

### Example 5: Analysis for 5-Hydroxytryptophan Expression Level

After subculture, rat pancreatic islet tumor RIN-14B cells were divided into three single-strain groups, three single-strain control groups, four multiple-strain groups, and three multiple-strain control groups. Additionally, the total bacterial counts for subsequent treatment in all groups were the same.

The three single-strain groups were named “BB-115 group”, “MP108 group”, and “BLI-02 group”, respectively. In the “BB-115 group”, cells were merely treated with a Bifidobacterium animalis subsp. lactis BB-115 strain; in the “MP108 group”, cells were merely treated with a Lactobacillus rhamnosus MP108 strain; in the “BLI-02 group”, cells were merely treated with a Bifidobacterium longum subsp. infantis BLI-02 strain.

The three single-strain control groups were named “HN019 group”, “LGG group”, and “M-63 group”, respectively. In the “HN019 group”, cells were merely treated with a Bifidobacterium animalis subsp. lactis HN019 strain; in the “LGG group”, cells were merely treated with a Lactobacillus rhamnosus LGG strain; in the “M-63 group”, cells were merely treated with a Bifidobacterium longum subsp. infantis M-63 strain.

The four multiple-strain groups were named “BB-115: MP108: BLI-02=1:1:1 group”, “BB-115: MP108: BLI-02=2:1:1 group”, “BB-115: MP108=1:1 group”, and “BB-115: BLI-02=1:1 group”, respectively. In the “BB-115: MP108: BLI-02=1:1:1 group”, cells were treated with a Bifidobacterium animalis subsp. lactis BB-115 strain, a Lactobacillus rhamnosus MP108 strain, and a Bifidobacterium longum subsp. infantis BLI-02 strain at a CFU ratio of 1:1:1; in the “BB-115: MP108: BLI-02=2:1:1 group”, cells were treated with a Bifidobacterium animalis subsp. lactis BB-115 strain, a Lactobacillus rhamnosus MP108 strain, and a Bifidobacterium longum subsp. infantis BLI-02 strain at a CFU ratio of 2:1:1; in the “BB-115: MP108=1:1 group”, cells were treated with a Bifidobacterium animalis subsp. lactis BB-115 strain and a Lactobacillus rhamnosus MP108 strain at a CFU ratio of 1:1; in the “BB-115: BLI-02=1:1 group”, cells were treated with a Bifidobacterium animalis subsp. lactis BB-115 strain and a Bifidobacterium longum subsp. infantis BLI-02 strain at a CFU ratio of 1:1.

The three multiple-strain control groups were named “HN019: MP108=1:1 group”, “HN019: BLI-02=1:1 group”, and “HN019: MP108: BLI-02=1:1:1 group”, respectively. In the “HN019: MP108=1:1 group”, cells were treated with a Bifidobacterium animalis subsp. lactis HN019 strain and a Lactobacillus rhamnosus MP108 strain at a CFU ratio of 1:1; in the “HN019: BLI-02=1:1 group”, cells were treated with a Bifidobacterium animalis subsp. lactis HN019 strain and a Bifidobacterium longum subsp. infantis BLI-02 strain at a CFU ratio of 1:1; in the “HN019: MP108: BLI-02=1:1:1 group”, cells were treated with a Bifidobacterium animalis subsp. lactis HN019 strain, a Lactobacillus rhamnosus MP108 strain, and a Bifidobacterium longum subsp. infantis BLI-02 strain at a CFU ratio of 1:1:1.

2×10⁵ cells were seeded into 0.5 mL of RPMI medium supplemented with 10% fetal bovine serum and 1% penicillin-streptomycin, and then placed in an incubator with 5% CO₂ at 37° C. for 72 hours. After removing the medium, the cells were washed twice with phosphate-buffered saline (PBS). Then, the corresponding bacterial strain samples were added to the cells at a total bacterial count of 10⁸ CFU and placed in an incubator with 5% CO₂ at 37° C. for 1 hour. On adding the bacterial strain samples, RPMI medium was added to the cells as a control group.

Enzyme-linked immunosorbent assay (ELISA) was used to analyze the cell culture medium obtained from every group. Specifically, the total 5-hydroxytryptophan level in the cell culture medium obtained from every group was measured according to the kit manufacturer's instructions. Based on the total 5-hydroxytryptophan level in the control group, the relative 5-hydroxytryptophan level in every group was calculated; that is,

$$relative 5 - hydroxytryptophan level in every group = \frac{total 5 - hydroxytryptophan level in every group}{total 5 - hydroxytryptophan level in control group} .$$

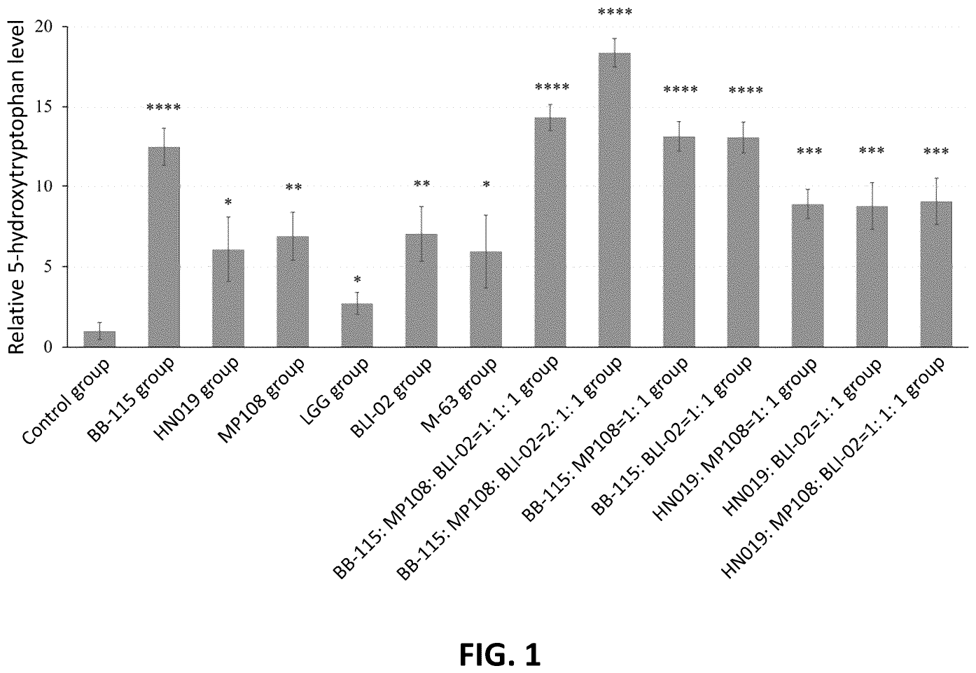

As shown in FIG. 1 and Table 2, although the BB-115 strain and the HN019 strain belong to the same species, Bifidobacterium animalis subsp. lactis, the BB-115 strain exhibits superior activity in promoting cells to secrete 5-hydroxytryptophan than the HN019 strain. Although the MP108 strain and the LGG strain belong to the same species, Lactobacillus rhamnosus, the MP108 strain exhibits superior activity in promoting cells to secrete 5-hydroxytryptophan than the LGG strain. Although the BLI-02 strain and the M-63 strain belong to the same species, Bifidobacterium longum subsp. infantis, the BLI-02 strain exhibits superior activity in promoting cells to secrete 5-hydroxytryptophan than the M-63 strain. The BB-115 strain promotes the secretion of 5-hydroxytryptophan in cells at least twice as effectively as the other strains. This indicates that the ability to promote the secretion of 5-hydroxytryptophan in cells is strain-specific.

As shown in FIG. 1 and Table 2, the combination of the BB-115 strain, the MP108 strain, and the BLI-02 strain, the combination of the BB-115 strain and the MP108 strain, and the combination of the BB-115 strain and the BLI-02 strain can exhibit more effective activity in promoting cells to secrete 5-hydroxytryptophan than the BB-115 strain alone. This indicates that the combination of the BB-115 strain and the MP108 strain and/or the BLI-02 strain can bring synergistic effects on promoting cells to secrete 5-hydroxytryptophan. Moreover, the combination of the BB-115 strain, the MP108 strain, and the BLI-02 strain can exhibit more effective activity in promoting cells to secrete 5-hydroxytryptophan than the combination of the BB-115 strain and the MP108 strain and the combination of the BB-115 strain and the BLI-02 strain.

As shown in FIG. 1 and Table 2, the combination of the HN019 strain, the MP108 strain, and the BLI-02 strain, the combination of the HN019 strain and the MP108 strain, and the combination of the HN019 strain and the BLI-02 strain can exhibit more effective activity in promoting cells to secrete 5-hydroxytryptophan than the HN019 strain alone. This indicates that the combination of the HN019 strain and the MP108 strain and/or the BLI-02 strain can bring synergistic effects on promoting cells to secrete 5-hydroxytryptophan. However, the combination of the HN019 strain, the MP108 strain, and the BLI-02 strain can exhibit activity in promoting cells to secrete 5-hydroxytryptophan equal to that of the combination of the HN019 strain and the MP108 strain and that of the combination of the HN019 strain and the BLI-02 strain. This indicates that the BB-115 strain can cooperate with the MP108 strain and the BLI-02 strain in synergistically promoting cells to secrete 5-hydroxytryptophan.

TABLE 2
relative 5-hydroxytryptophan level

| | Average of relative 5- |
|---|---|
| Group | hydroxytryptophan level |
| Control group | 1.0 |
| BB-115 group | 12.50 |
| HN019 group | 6.08 |
| MP108 group | 6.89 |
| LGG group | 2.71 |
| BLI-02 group | 7.03 |
| M-63 group | 5.94 |
| BB-115:MP108:BLI-02 = 1:1:1 group | 14.33 |
| BB-115:MP108:BLI-02 = 2:1:1 group | 18.36 |
| BB-115:MP108 = 1:1 group | 13.15 |
| BB-115:BLI-02 = 1:1 group | 13.08 |
| HN019:MP108 = 1:1 group | 8.89 |
| HN019:BLI-02 = 1:1 group | 8.75 |
| HN019:MP108:BLI-02 = 1:1:1 group | 9.07 |

As described above, a Bifidobacterium animalis subsp. lactis BB-115 strain has a good ability to enhance the secretion of 5-hydroxytryptophan by enterochromaffin cells. Moreover, in combination with a Lactobacillus rhamnosus MP108 strain and a Bifidobacterium longum subsp. infantis BLI-02 strain, it can effectively and synergistically increase the ability of 5-hydroxytryptophan secretion by enterochromaffin cells. Accordingly, it is expected to be useful in addressing emotional issues, such as insomnia, depression, anxiety, and lack of concentration.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A method for enhancing 5-hydroxytryptophan secretion by enterochromaffin cells, comprising:
administering a composition to a subject in need thereof;
wherein the composition comprises: a Bifidobacterium animalis subsp. lactis BB-115 strain deposited at the China General Microbiological Culture Collection Center with a deposition number of CGMCC 21840.

2. The method as claimed in claim 1, wherein the Bifidobacterium animalis subsp. lactis BB-115 strain is an active strain or an inactivated strain.

3. The method as claimed in claim 1, being further for enhancing immunity, treating insomnia, treating depression, treating hypertension, improving somnipathy, improving anxiety, improving inattention, treating menopausal syndrome, or treating gastrointestinal disease.

4. The method as claimed in claim 1, wherein the composition is a medicine, a food, or a health supplement.

5. A method for enhancing 5-hydroxytryptophan secretion by enterochromaffin cells, comprising:
administering a composition to a subject in need thereof;
wherein the composition comprises: a Bifidobacterium animalis subsp. lactis BB-115 strain deposited at the China General Microbiological Culture Collection Center with a deposition number of CGMCC 21840, a Lactobacillus rhamnosus MP108 strain deposited at the China General Microbiological Culture Collection Center with a deposition number of CGMCC 21225, and a Bifidobacterium longum subsp. infantis BLI-02 strain deposited at the China General Microbiological Culture Collection Center with a deposition number of CGMCC 15212.

6. The method as claimed in claim 5, wherein the Bifidobacterium animalis subsp. lactis BB-115 strain is an active strain or an inactivated strain.

7. The method as claimed in claim 5, wherein the Lactobacillus rhamnosus MP108 strain is an active strain or an inactivated strain.

8. The method as claimed in claim 5, wherein the Bifidobacterium longum subsp. infantis BLI-02 strain is an active strain or an inactivated strain.

9. The method as claimed in claim 5, wherein a CFU (colony forming unit) ratio of the Bifidobacterium animalis subsp. lactis BB-115 strain to the Lactobacillus rhamnosus MP108 strain to the Bifidobacterium longum subsp. infantis BLI-02 strain is (1 to 2): 1:1.

10. The method as claimed in claim 5, wherein a CFU (colony forming unit) ratio of the Bifidobacterium animalis subsp. lactis BB-115 strain to the Lactobacillus rhamnosus MP108 strain to the Bifidobacterium longum subsp. infantis BLI-02 strain is 2:1:1.

11. The method as claimed in claim 5, being further for enhancing immunity, treating insomnia, treating depression, treating hypertension, improving somnipathy, improving anxiety, improving inattention, treating menopausal syndrome, or treating gastrointestinal disease.

12. The method as claimed in claim 5, wherein the composition is a medicine, a food, or a health supplement.
